(19) United States
(12) Patent Application Publication
(10) Pub. No.: US 2004/0236342 A1
(43) Pub. Date: Nov. 25, 2004

(54) Device to assess ADR motion

(76) Inventors: Bret A. Ferree, Cincinnati, OH (US); David Tompkins, Milford, OH (US); Alex B. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/876,070

(22) Filed: Jun. 24, 2004

Related U.S. Application Data
(63) Continuation-in-part of application No. 10/421,436, filed on Apr. 23, 2003.
(60) Provisional application No. 60/374,747, filed on Apr. 23, 2002.
(60) Provisional application No. 60/484,935, filed on Jul. 3, 2003.
(60) Provisional application No. 60/519,045, filed on Nov. 11, 2003.
(60) Provisional application No. 60/530,579, filed on Dec. 18, 2003.

(51) Int. Cl. A61B 17/60; A61B 17/70

(52) U.S. Cl. 606/102; 606/105; 606/86; 606/61

(57) ABSTRACT

Devices and associated methods help surgeons assess spinal and artificial disc replacement (ADR) position, orientation and motion. The device moves the spine through flexion, extension, lateral bending, and/or axial rotation while measuring the amount of movement in these directions. The device may also measures the force required to move the spine in one or more of the above-mentioned directions. The various disclosed embodiments maximize motion through careful alignment, size, location in the disc space, configuration of the articulating surfaces of the ADR, and adequate soft tissue release. The invention may utilizes pre-operative images to determine the preferred alignment of the ADR; intra-operative images to align the instruments, trial ADRs and the ADR; devices that assess the ROM of the vertebrae after soft tissue release; devices that determine the proper size of the ADR; devices that test the motion after machining the vertebrae; or ADR embodiments with different degrees of axial rotation. Although the various procedures are described for ADRs that are inserted from an anterior approach to the spine, the invention could also be used for ADRs that are inserted from a lateral or posterior-lateral approach to the spine.

### REFERENCE TO RELATED APPLICATION

This application claims priority from U.S. Provisional Patent Application Ser. No. 60/484,935, filed Jul. 3, 2003; 60/519,045, filed Nov. 12, 2003; and 60/530,579, filed Dec. 18, 2003.

This application is also a continuation-in-part of U.S. patent application Ser. No. 10/421,436, filed Apr. 23, 2003, which claims priority from U.S. Provisional Patent Application Ser. No. 60/374,747, filed Apr. 23, 2002. The entire content of each application is incorporated herein by reference.

### FIELD OF THE INVENTION

This invention relates generally to spinal surgery and, in particular, to devices and methods used to assess spinal motion; artificial disc replacement strategies; modular articulating components; and alignment optimization.

### BACKGROUND OF THE INVENTION

Premature or accelerated intervertebral disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures, either remove the disc or the disc's function by connecting two or more vertebra together.

These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, replace either the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant.

### SUMMARY OF THE INVENTION

According to one aspect of this invention, devices and associated methods are disclosed for assessing spinal and artificial disc replacement (ADR) motion. In some embodiments, such devices are used to assess spinal motion before the insertion of an ADR. The invention thus helps surgeons determine if additional soft tissue release is needed before ADR insertion. The device moves the spine through flexion, extension, lateral bending, and/or axial rotation while measuring the amount of movement in these directions. The device may also measures the force required to move the spine in one or more of the above-mentioned directions.

A different embodiment of the invention attaches to an implanted ADR to measure the amount of motion that the ADR allows. The device may also measure the forces required to move the ADR in one or more directions. Using the information provided by the device, a surgeon may select an ADR of a different size in an attempt to improve spinal motion. Fluoroscopy, x-ray, or other navigation device may be used to help assess spinal and ADR motion.

ADR motion may be determined by several factors including, ADR size, the configuration of the articulating surfaces of the ADR, the extent of the release of the soft tissues about the spine, ADR placement, and ADR alignment. The methods and devices taught in this application maximize the range of motion of implanted ADRs. Previous studies have shown that ADRs that do not move well in vivo lead to accelerated disc degeneration of the discs adjacent to the ADR.

Generally speaking, the inventions disclosed in this application maximize the motion of ADRs through careful ADR alignment, size, location in the disc space, configuration of the articulating surfaces of the ADR, and adequate soft tissue release. The invention may utilize pre-operative images to determine the preferred alignment of the ADR; intra-operative images to align the instruments, trial ADRs and the ADR; devices that assess the ROM of the vertebrae after soft tissue release; devices that determine the proper size of the ADR; devices that test the motion after machining the vertebrae; or ADR embodiments with different degrees of axial rotation. Although the various procedures are described for ADRs that are inserted from an anterior approach to the spine, the invention could also be used for ADRs that are inserted from a lateral or posterior-lateral approach to the spine. The devices may need to be modified for use with these different approaches.

### BRIEF DESCRIPTION OF THE DRAWINGS

FIG. 1 is a sagittal cross-section of the spine of the device of the present invention;

FIG. 2 is a sagittal cross-section of the spine and an alternative embodiment of the device;

FIG. 3 is a sagittal cross-section of the spine, an ADR, and an alternative embodiment of the device that removably attaches to an ADR;

FIG. 4A is an anterior view of an alternative embodiment of the present invention;

FIG. 4B is a lateral view of the embodiment of the invention drawn in FIG. 4A;

FIG. 4C is an anterior view of a trial spacer;

FIG. 4D is a sagittal cross section of the trail spacer drawn in FIG. 4C and the endplates of the device drawn in FIG. 4B;

FIG. 4E is an anterior view of modular articulating components;

FIG. 4F is an anterior view of the embodiment of the invention drawn in FIG. 4A;

FIG. 4G is a lateral view of the embodiment of the invention drawn in FIG. 4F;

FIG. 4H is an anterior view of the embodiment of the invention drawn in FIG. 4G;

FIG. 41 is a sagittal cross-section of the embodiment of the invention drawn in FIG. 4F;

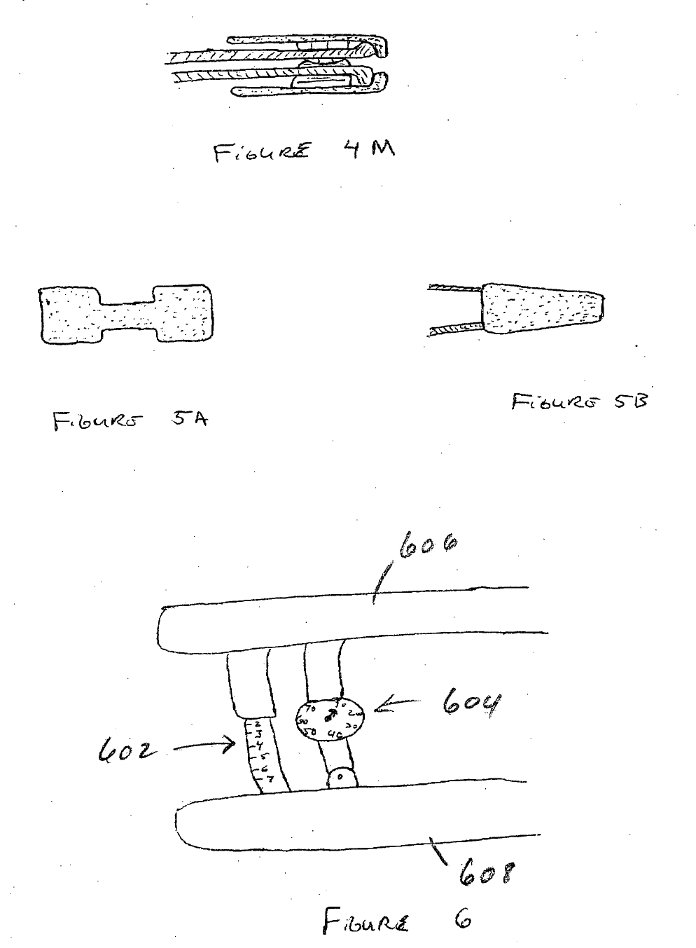

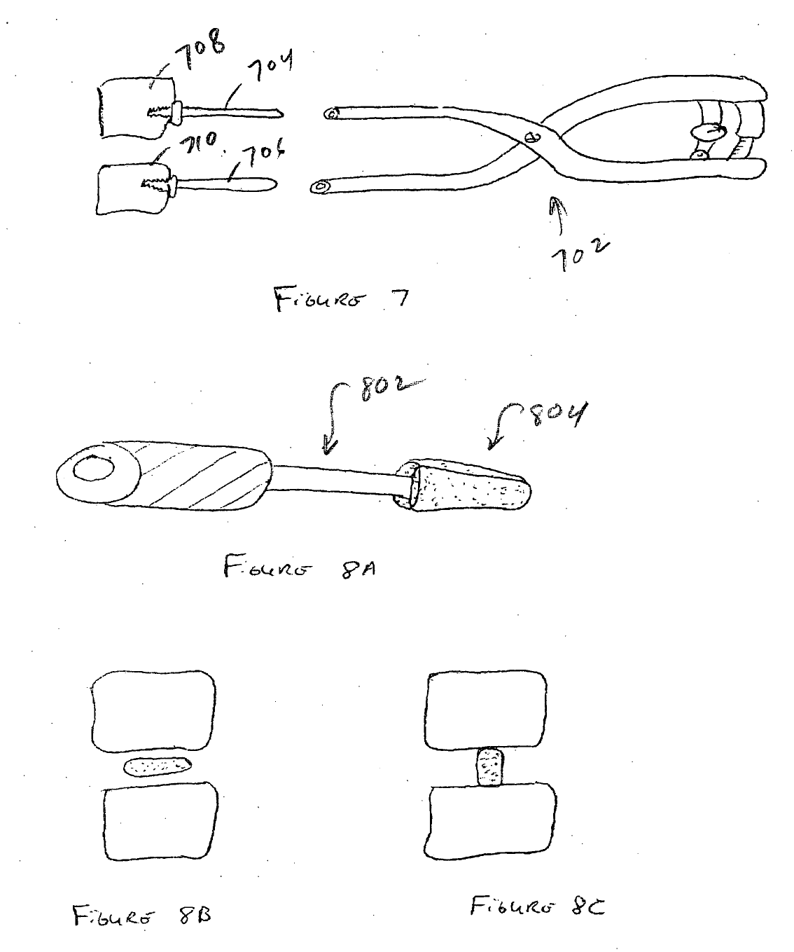

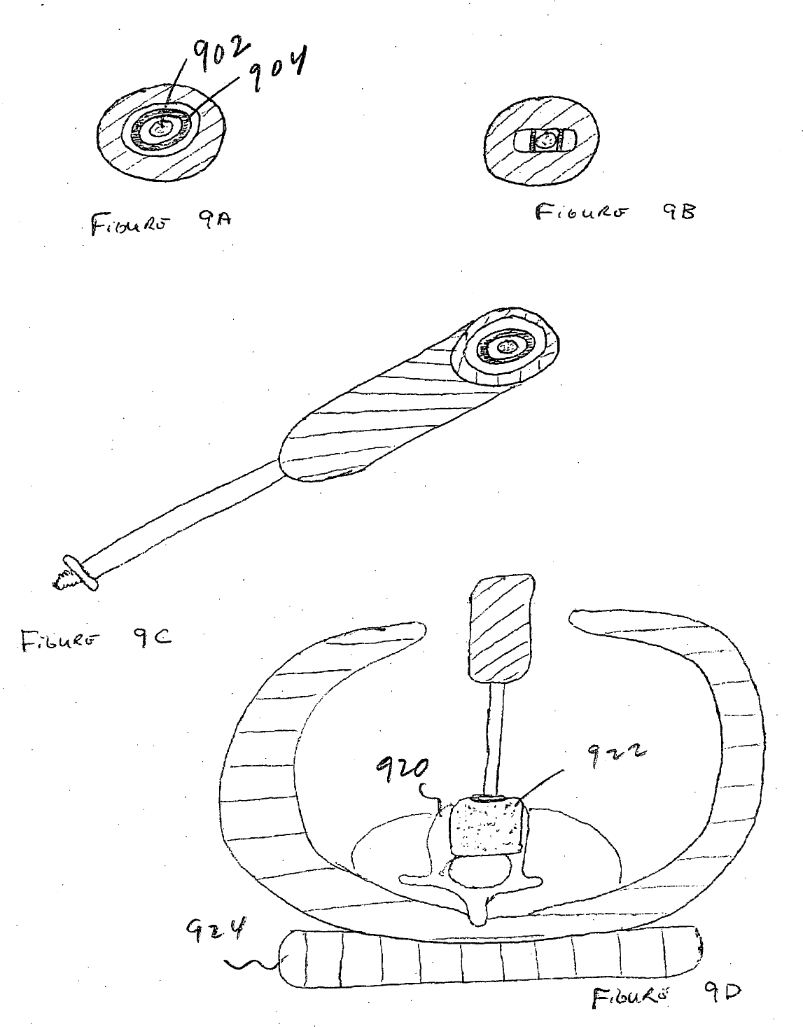

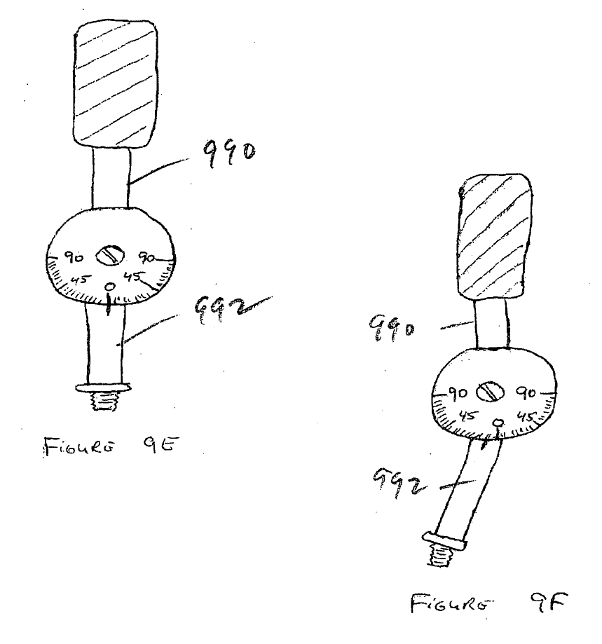

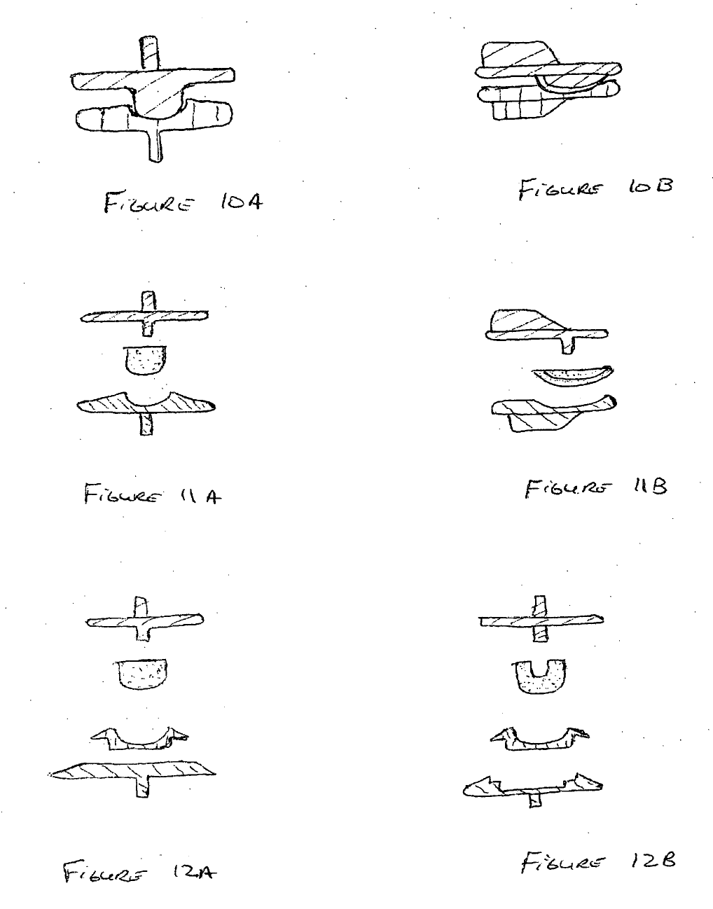

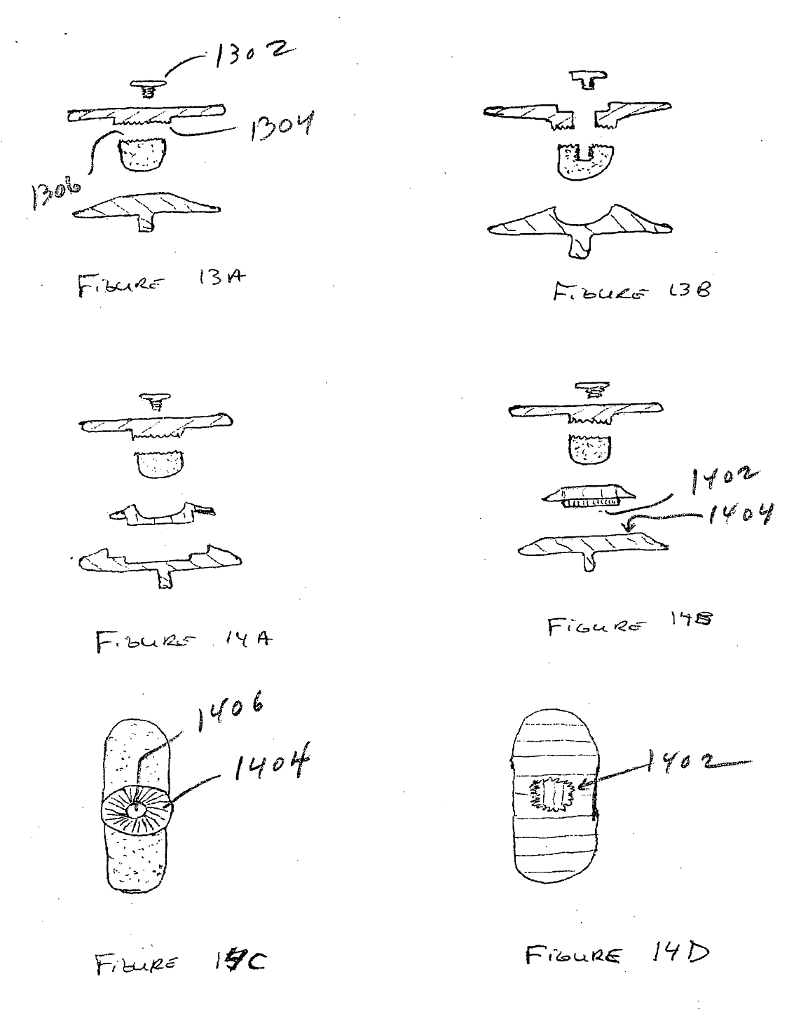

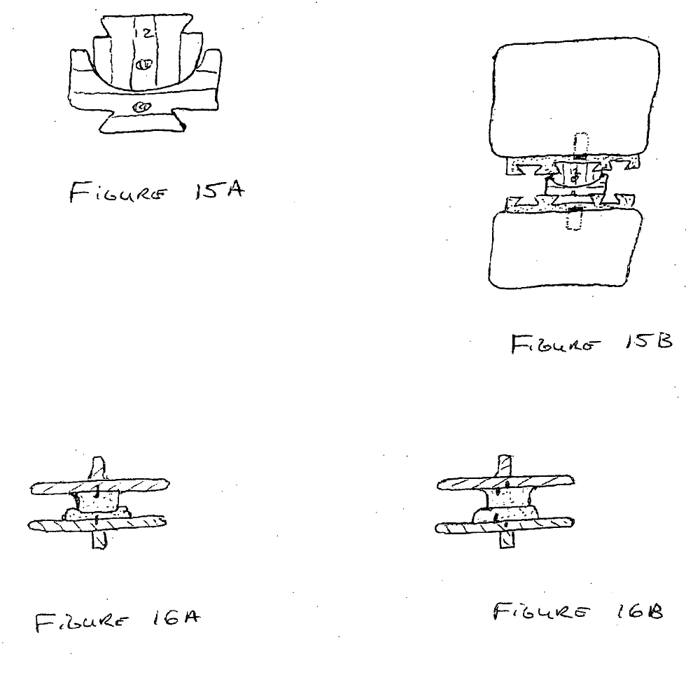

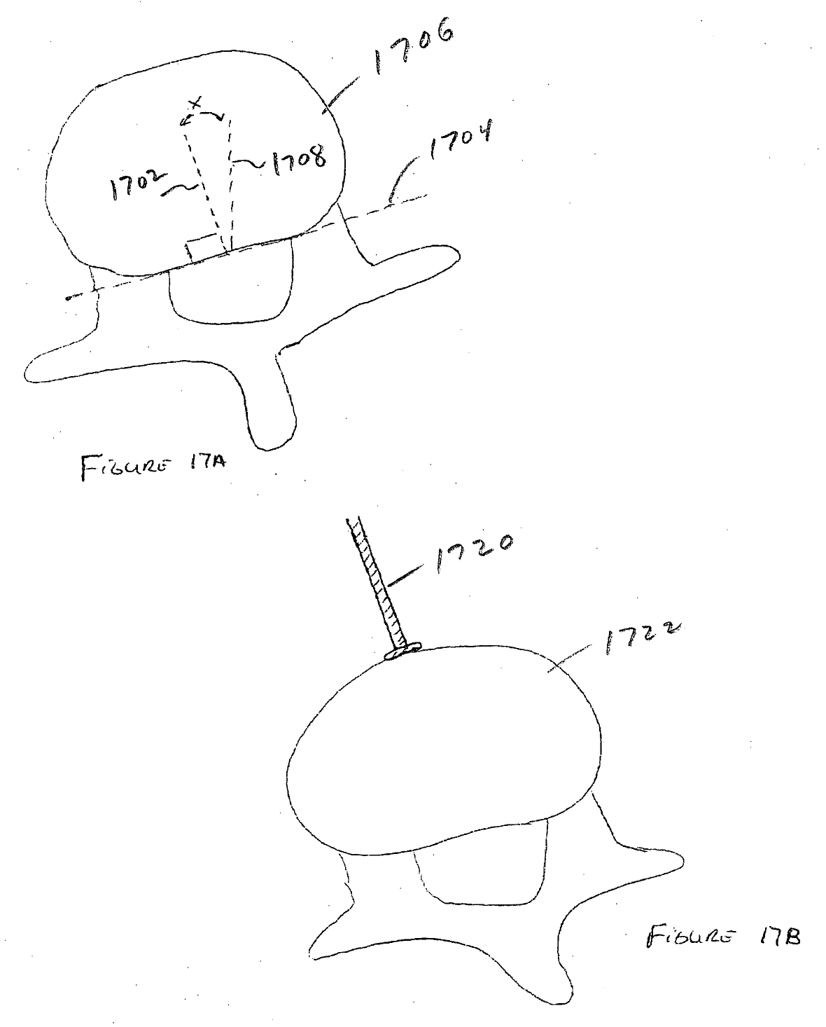

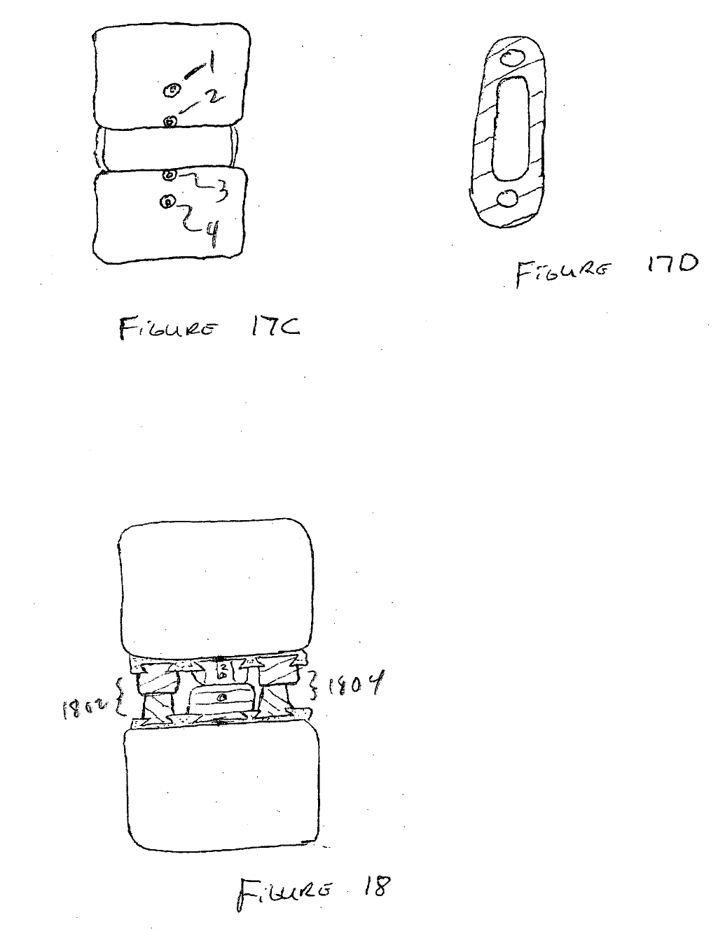

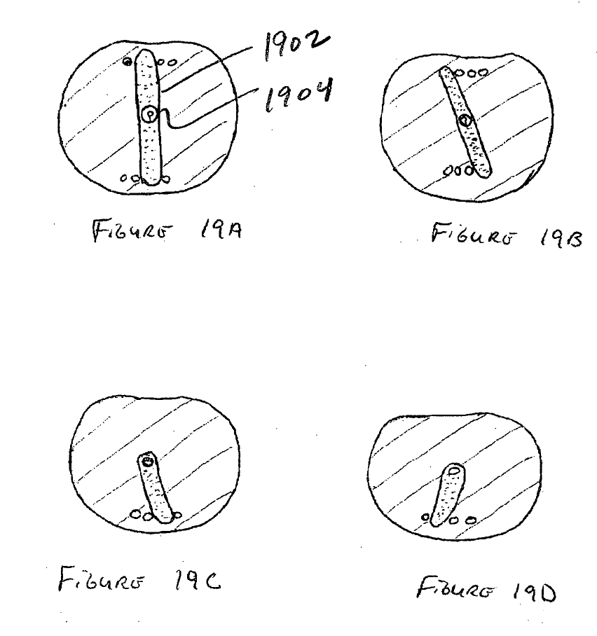

FIG. 4J is a sagittal cross-section of the embodiment of the invention drawn in FIG. 4G and an alternative embodiment of the instrument used to flex the device;

FIG. 4K is an anterior view of the embodiment of the invention drawn in FIG. 4H;

FIG. 4L is an anterior view of the embodiment of the device drawn in FIG. 4K;

FIG. 4M is a sagittal cross section of the device drawn in FIG. 4J and an alternative embodiment of the arms of the instrument drawn in FIG. 4J;

FIG. 5A is an anterior view of an alternative embodiment of the present invention;

FIG. 5B is a lateral view of the embodiment of the invention drawn in FIG. 5A;

FIG. 6 is a lateral view of the handles of the distraction and compression instruments;

FIG. 7 is a partial sagittal cross-section of the spine and an alternative embodiment of the instrument;

FIG. 8A is a lateral view of an alternative embodiment of the invention;

FIG. 8B is an anterior view of the spine and a cross section of the distraction component drawn in FIG. 8A;

FIG. 8C is an anterior view of the spine and the embodiment of the invention drawn in FIG. 8B;

FIG. 9A is a view of the end of the handle of a surgical instrument of an alternative embodiment of the present invention;

FIG. 9B is a view of the end of the handle of a surgical instrument with an alternative embodiment of the level drawn in FIG. 9A;

FIG. 9C is an oblique view of a surgical instrument and the embodiment of the level drawn in FIG. 9A;

FIG. 9D is an axial cross section of the body, a disc, an ADR, an OR table, and the instrument drawn in FIG. 9A;

FIG. 9E is a lateral view of an alternative embodiment of the invention device drawn in FIG. 9C;

FIG. 9F is a lateral view of the embodiment of the device drawn in FIG. 9E;

FIG. 10A is a coronal cross-section of a novel ADR;

FIG. 10B is a sagittal cross-section of the ADR drawn in FIG. 10A;

FIG. 11A is an exploded anterior view of an alternative embodiment of the ADR drawn in FIG. 10A;

FIG. 11B is a sagittal cross section of the embodiment of the invention drawn in FIG. 11A;

FIG. 12A is an exploded, partial coronal cross-section of an alternative embodiment of the ADR drawn in FIG. 11A;

FIG. 12B is an exploded coronal cross section of the embodiment of the invention drawn in FIG. 12A;

FIG. 13A is an exploded anterior view of an alternative embodiment of the ADR drawn in FIG. 11A;

FIG. 13B is an exploded coronal cross section of the embodiment of the invention drawn in FIG. 13A;

FIG. 14A is an exploded coronal cross section of an alternative embodiment of the ADR drawn in FIG. 14A;

FIG. 14B is an exploded coronal cross-section of an alternative embodiment of the ADR drawn in FIG. 14A;

FIG. 14C is a view of the top of the convex component drawn in FIG. 13A;

FIG. 14D is a view of the bottom of the concave articulating component drawn in FIG. 14B;

FIG. 15A is a coronal cross section of an alternative embodiment of the modular articulating components drawn in FIG. 4E;

FIG. 15B is an anterior view of the spine, the articulating components drawn in FIG. 15A, and the device drawn in FIG. 4H;

FIG. 16A is an anterior view of an alternative embodiment of the invention;

FIG. 16B is an anterior view of the trial ADR drawn in FIG. 16A;

FIG. 17A is an axial cross section through the upper vertebral endplate (VEP) of a vertebra;

FIG. 17B is an axial cross-section of a vertebra;

FIG. 17C is an anterior view of the spine;

FIG. 17D is an anterior view of one embodiment of an instrument that cooperates with the guide pins drawn in FIG. 17C;

FIG. 18 is an anterior view of the spine and an alternative embodiment of the invention drawn in FIG. 15B;

FIG. 19A is a superior view of an ADR EP of an alternative embodiment of the invention;

FIG. 19B is a superior view of the ADR EP drawn in FIG. 19A;

FIG. 19C is a superior view of an ADR EP of an alternative embodiment of the invention drawn in FIG. 19A; and

FIG. 19D is a superior view of the ADR EP drawn in FIG. 19C.

### DETAILED DESCRIPTION OF THE INVENTION

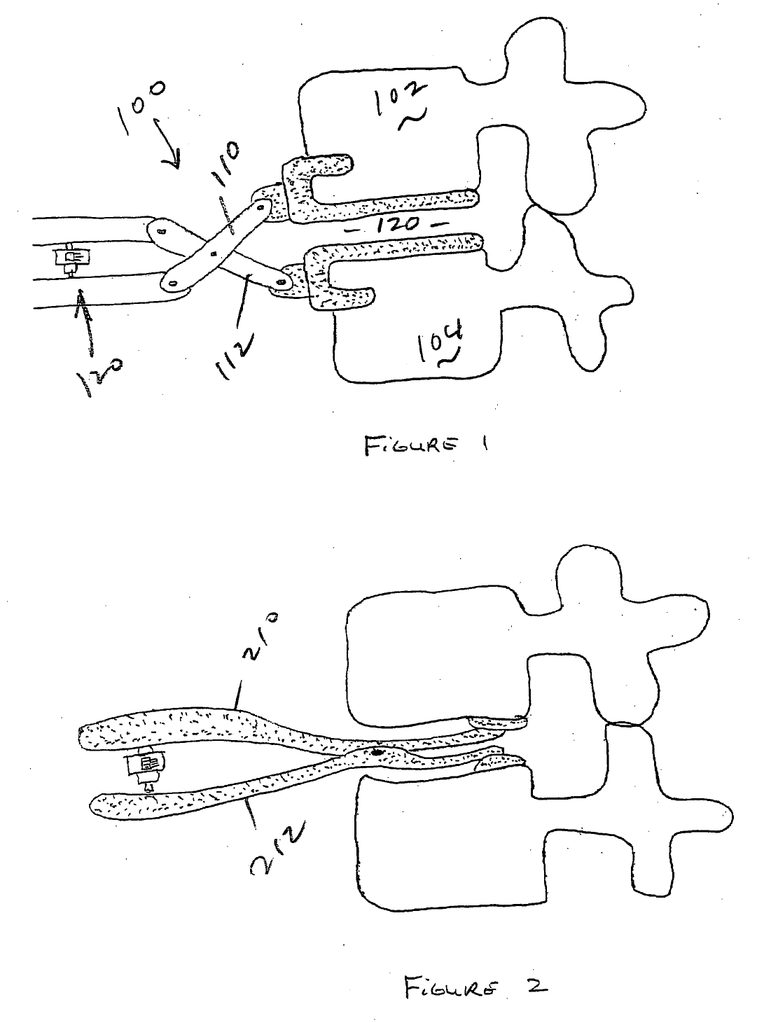

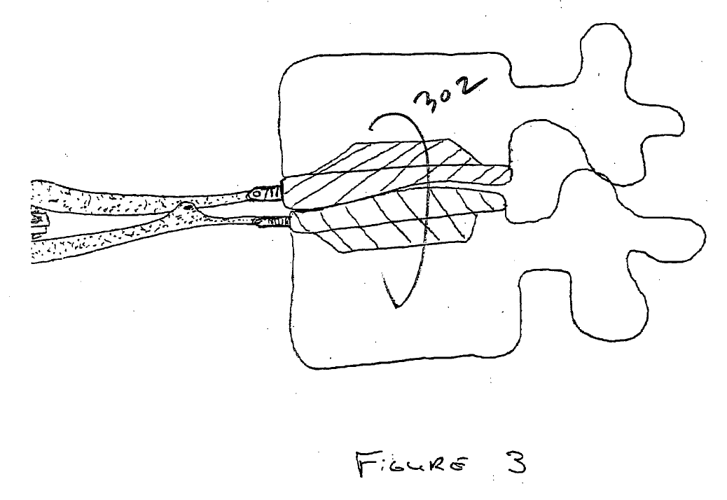

FIG. 1 is a sagittal cross section of the spine and the device 100 according to the invention that attaches to the front of vertebrae 102, 104. Arms 110, 112 from the device extend into the disc space 120. The spine is forced into flexion by pulling the anterior portion of the device together. A spacer can be placed between posterior portion of the intradiscal arms of the device to facilitate spinal flexion.

Spinal extension is reproduced by distracting the anterior portion of the vertebrae. Lateral bending is reproduced by distracting the lateral side of the disc space. Axial rotation can be assessed by rotating the arms of the device in opposite directions. A device 120 is used to measures the amount of spinal movement and the force required to produce a particular movement. The device 120 may be mechanical, as shown with a spring-loaded graduated cylinder, or electronic, using a piezoelectric material, strain gauge or other component interfaced to appropriate electronics well known to those of skill in the art of position and pressure sensing, and the like.

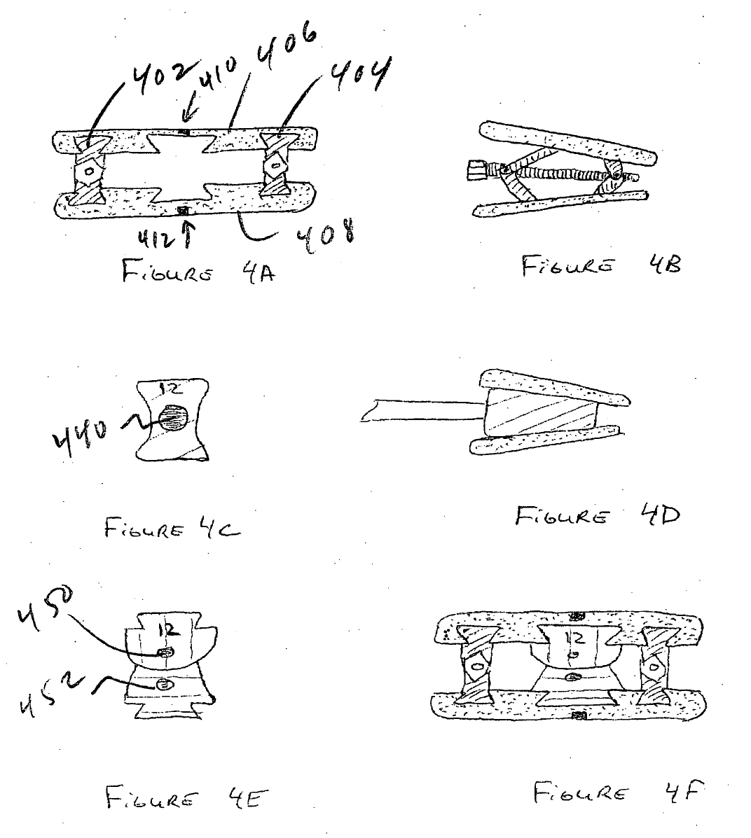

FIG. 2 is a sagittal cross section of the spine and an alternative embodiment of the device. Intradiscal arms 210, 212 may be placed at various locations within the disc space to reproduce spinal motion. The drawing illustrates placement of the arms of the device in the posterior aspect of the disc space to help assess spinal flexion. The device can also be used to assess extension and lateral bending. The device determines the amount of motion in each direction, as well as the force required to produce the motion.

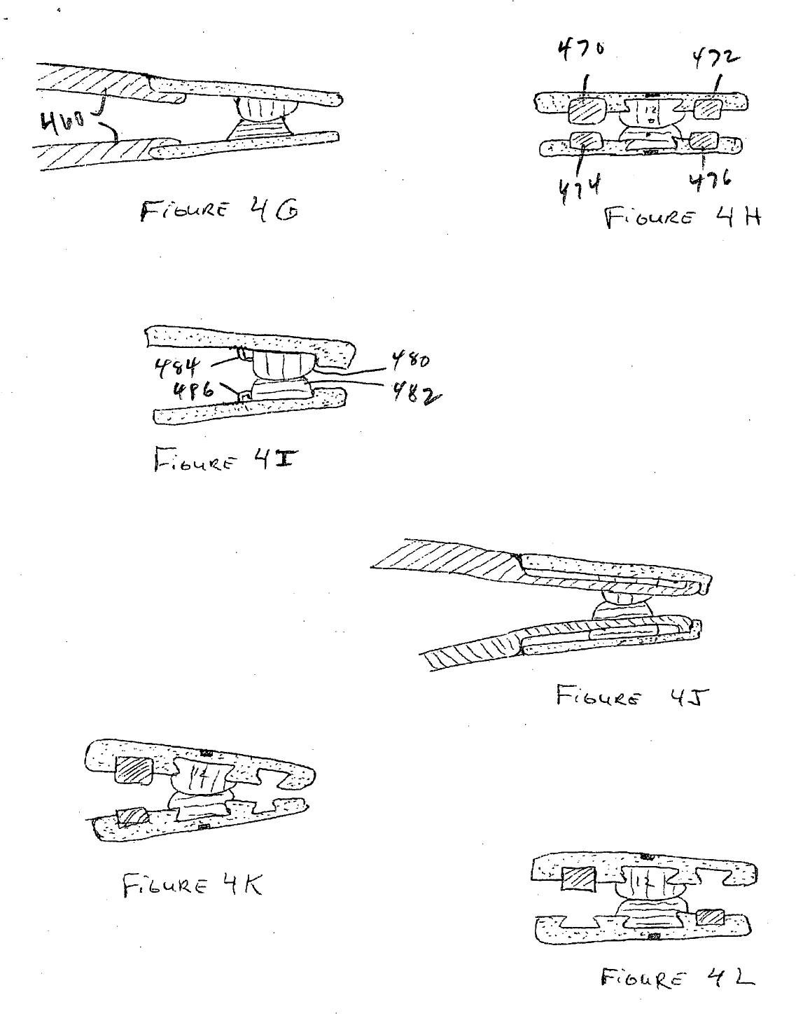

FIG. 3 is a sagittal cross section of the spine, an ADR, and an alternative embodiment of the device that removably attaches to an ADR 302. Once attached to the ADR, the device causes the ADR and the spine to flex, extend, bend laterally, and rotate in an axial direction. The amount of movement in each direction, and the force required to produce the movement is measured by the device 330.

FIG. 4A is an anterior view of an alternative embodiment of the invention. The device is used to distract the disc space. The drawing shows scissors jacks 402, 404 that are used to distract metal endplates 406, 408. The device is inserted into the disc space in a collapsed configuration. Unlike prior-art impacted distractors, the device does not subject the vertebral endplates (VEPs) to shear forces. Impacted distractors risk damage and fractures of the VEPs. This embodiment of the invention is related to the distraction sleeves taught in my co-pending U.S. patent application Ser. No. 10/421,436, the entire content of which is incorporated herein by reference.

The scissor jacks 402, 404 may be extended with a torque wrench or a torque screwdriver. The torque could be selected based upon a patient's age, sex, size, and bone quality. Extension of the scissor jacks with torque wrenches helps prevent fractures of the VEPs. The upper and lower plates of the device have marks 410, 412 to identify the midline of the device. FIG. 4B is a lateral view of the embodiment of the invention drawn in FIG. 4A.

FIG. 4C is an anterior view of a trial spacer according to the invention. The circle 440 represents the shaft of an instrument used to place the spacer. The number indicates the size of the spacer, for example, 12 mm. The spacer is used to measure the size of the opening between the distracted endplates of the device.

FIG. 4D is a sagittal cross section of the trail spacer drawn in FIG. 4C and the endplates of the device drawn in FIG. 4B. FIG. 4E is an anterior view of modular articulating components. The circles 450, 452 on the components represent surface irregularities that cooperate with an instrument or instruments used to insert the articulating components. The preferred embodiment of the device uses modular ball and socket components. Other types of articulating surfaces may be used in this embodiment of the device.

The modular components may be impacted between the endplates of the device drawn in FIG. 4A. Impacting the articulating components between the endplates distracts the disc space. Note that the scissor jacks may not be required if the articulating components are impacted between the endplate's. FIG. 4F is an anterior view of the embodiment of the invention drawn in FIG. 4A. The modular articulating components of FIG. 4E have been inserted into slots of the device. The sizes of the modular articulating components were determined by use of the spacer drawn in FIG. 4C. The scissor jacks are removed after the articulating components are inserted.

FIG. 4G is a lateral view of the embodiment of the invention drawn in FIG. 4F with the scissor jacks removed. An instrument 460 is used to move the assembled device through a range of motion. The arms of the instrument may fit into the slots that receive the scissor jacks or the articulating components. Other mechanisms of coupling the instruments and the device may be used. As discussed with reference to FIG. 1, the instrument records the amount the ADR has moved and the force required to move the ADR. For example, the instrument may record the number of millimeters the device has moved and the force in inch/pounds required to move the device. The instrument may also record the degrees the device has moved.

The instrument or instruments preferably move the device through flexion, extension, lateral bending, and axial rotation. The instrument may distract the anterior portion of the device to test extension of the device. The instrument may compress the anterior portion of the device to test flexion of the device. The arms of the instrument may be trapezoidal in cross section to fit in the slots of the device that are also trapezoidal in cross section. Lateral bending may be tested by compression and/or distraction of one or both sides of the device. The instrument may be connected to a microprocessor controlled monitor. The monitor may record the total degrees of motion the device traveled in each plane or axis of rotation. FIG. 4H is an anterior view of the embodiment of the invention drawn in FIG. 4G. The arms of the compression or distraction instrument are seen in cross section (area 470, 472, 474, 476). The instrument drawn in FIG. 4H is used to test flexion and extension of the device.

FIG. 4I is a sagittal cross section of the embodiment of the invention including modular articulating components 480, 482 spring-loaded projections 484, 486 that fit into recesses in the slots of the device. The spring-loaded projections 480, 482 hold the articulating components within the endplates of the device. Other mechanisms of coupling the articulating components and the endplates of the device are possible. FIG. 4J is a sagittal cross section of an alternative embodiment of the instrument used to flex the device. The arms of the instrument distract the posterior portion of the device to cause the device to flex.

FIG. 4K is an anterior view of the embodiment of the invention that shows lateral bending to the device by the arms of a distraction instrument. A compression instrument may be used to test lateral bending in the opposite direction. Alternatively, the distraction instrument may be moved to the contra-lateral side of the device to test lateral bending in the opposite direction. One embodiment of the invention uses the same instrument that compresses, distracts, and records the values for both compression and distraction.

FIG. 4L is an anterior view of the embodiment of the device drawn in FIG. 4K, wherein the arms of the instrument are used to test and record axial rotation of the device. FIG. 4M is a sagittal cross section of the device drawn in FIG. 4J and an alternative embodiment wherein the arms of the distraction instrument have a reduced profile.

FIG. 5A is an anterior view of a device impacted between the vertebrae to distract the disc space. The recesses in the top and the bottom of the device are designed to receive the arms of an instrument. FIG. 5B is a lateral view of the embodiment of the invention drawn in FIG. 5A. The arms of a distractor instrument have been inserted into the recesses of the device. The distractor instrument records the force required to further distract the disc space.

FIG. 6 is a lateral view of the handles of the distraction and compression instruments. The drawing depicts one embodiment of the components use measure distance or degrees of travel of devices such as that drawn in FIG. 4G and the force required to generate the movement. A first set of components 602 measures distance or degrees traveled, whereas a second set of components 604 records the force exerted on the handles 606, 608 of the instrument. Other information such as date, time, and so forth, may also be fed to scales on top of the device or to a separate monitor.

Surgeons may use the information provided by this invention to change the size of the ADR, the type of ADR, the position of the ADR, the alignment of the ADR, or the extent of soft tissue release. The invention taught in my co-pending U.S. patent application Ser. No. 10/410,026, incorporated herein by reference, maybe combined with the teachings disclosed herein; for example, the surgeon may elect to use a particular type of ADR, in the size determined by the device of FIG. 4G, in the same alignment and position of the device of FIG. 4G, if the device of FIG. 4G moved through an acceptable range of motion with an acceptable amount of force and that remained in the disc space with an acceptable amount of pull by the device described in Ser. No. 10/410,026. The surgeon may elect to change one of the variables (ADR type, ADR size, ADR position, ADR alignment, or soft tissue release) if the device of FIG. 4G moved too little or required too much force to move.

FIG. 7 is a partial sagittal cross section of the spine and a distraction/compression instrument 702 that may be placed over the shafts of screws 704, 706 inserted into vertebral bodies 708, 710. The device measures the amount of vertebral movement and the force required to produce the movement.

FIG. 8A is a lateral view of an alternative embodiment of the invention in the form of a torque wrench or torque screwdriver 802 that is attached to a distraction component 804. FIG. 8B is an anterior view of the spine and a cross section of the distraction component drawn in FIG. 8A. The distraction component is drawn in horizontal position. FIG. 8C is an anterior view of the spine and the embodiment of the invention drawn in FIG. 8B. The distraction component has been rotated 90 degrees to “cam” open the disc space. The torque wrench determines the force required to “cam” open or distract the disc space.

FIG. 9A is a view of the end of the handle of a surgical instrument incorporating a bubble level. The circle 902 represents a gas bubble. The dark ring 904 outside the bubble represents the target for the bubble. The level helps the surgeon align the instrument. FIG. 9B is a view of the end of the handle of a surgical instrument with an alternative embodiment of a level. FIG. 9C is an oblique view of a surgical instrument and the embodiment of the level drawn in FIG. 9A.

FIG. 9D is an axial cross section of a human body, a vertebrae 920, an ADR 922, an OR table 924, and the instrument drawn in FIG. 9A. Using the levels disclosed herein, a surgeon can assure his instrument, and the attached ADR, is perpendicular to the OR table. Thus, as long as the patient is lying properly on the OR table, and the patient does not have a rotational abnormality of the spine, the novel instrument assures the ADR is placed with the proper rotational alignment. The normal disc allows only 1-2 degrees of axial rotation. ADRs that permit excessive axial rotation may damage the Annulus Fibrosus (AF) or the facet joints. Thus, some ADR designs limit axial rotation.

ADRs that limit axial rotation must be aligned properly or the ADR will not move properly. Mal-alignment of ADRs that limit axial rotation will increase the forces required to move the ADR in any direction. This and other embodiments of the invention help surgeons align ADRs to maximize in vivo movements of the ADR. The invention also relies on measurements from pre-operative imaging studies such as x-rays, CT scans, and MRI scans coupled with intra-operative images from Fluoroscopy, CT scans, or MRI scans to maximize ADR placement and ADR alignment. By way of example, FIG. 17A shows the measurement of the axial rotation of the spine from a pre-operative CT or MRI scan. ADRs that limit axial rotation should be placed into the disc space with the same axial alignment of the disc that is being replaced. The device drawn in FIG. 9D is used to align ADRs when the patient does not have rotational abnormalities of their spine.

FIG. 9E is a lateral view of an alternative embodiment of a device used to insert ADRs into the disc spaces of patients with axial rotation of their spines. For example, if measurement of a pre-operative CT scan shows the patient's disc is rotated 5 degrees to the right, the device may be used to insert the ADR with 5 degrees of axial rotation to the right. The device is temporarily locked with the shaft components 990, 992 angled 5 degrees relative to one another. FIG. 9F is a lateral view of the embodiment of the device drawn in FIG. 9E. The instrument has been locked to provide the proper axial rotation alignment. The locked instrument provides the proper axial rotational alignment when the bubble is centered within the handle of the device and the patient is lying flat on the OR table.

FIG. 10A is a coronal cross section of an ADR according to the invention which has limited axial rotation, flexion, extension, and lateral bending. The elongated convex projection from the upper ADR Endplate. (ADR EP) articulates in an elongated concavity in the lower ADR EP. The articulating components are preferably congruent; that is, they feature the same radius of curvature and maintain area contact throughout the range of motion between the components. The articulating components are incongruent in an alternative embodiment of the ADR. The lateral sides of the ADR EP also preferably impinge to limit lateral bending. The posterior portions of the ADR EPs may also impinge to limit extension. The anterior portions of the ADR EP may further impinge to limit flexion. The sides of the elongated convex component may impinge against the walls of the elongated concave component to limit axial rotation. Also preferably, the articulating surface of the concavity is larger than the articulating surface of the convexity.

FIG. 10B is a sagittal cross section of the ADR drawn in FIG. 10A. In the preferred embodiment of the device, the same radius used to create the curvature from anterior to posterior of the articulating surfaces of the ADR is the same as the radius used to create the curvature from the left to the right of the articulating surfaces of the ADR.

FIG. 11A is an exploded anterior view of an alternative embodiment of the ADR drawn in FIG. 10A. This embodiment of the ADR incorporates certain features taught in my co-pending U.S. Patent Application Ser. No. 60/518,971, incorporated herein by reference. The modular convex component enjoys unrestricted axial rotation around a post from the upper ADR EP. FIG. 1B is a sagittal cross section of the embodiment of the invention drawn in FIG. 11A.

FIG. 12A is an exploded, partial coronal cross section of an alternative embodiment of the ADR drawn in FIG. 11A. The modular concave enjoys unrestricted axial rotation in a concavity within the lower ADR EP. FIG. 12B is an exploded coronal cross section of the embodiment of the invention drawn in FIG. 12A.

FIG. 13A is an exploded anterior view of an alternative embodiment wherein the axial rotation of the convex component may be adjusted and fixed relative to the axial rotation of the upper ADR EP. FIG. 13B is an exploded coronal cross section of the embodiment of the invention drawn in FIG. 13A. A screw is used to attach the convex component to the upper ADR EP. The screw 1302 also holds the interdigitating teeth between the convex component 1304 and the upper ADR EP 1306 together. This embodiment of the invention allows a surgeon to change the axial alignment of one of the articulating components relative to one of the ADR EPs. For example, if the surgeon has cut slots in the vertebrae, but an inserted trial ADR does not move well when the trial is inserted into the machined vertebrae, the axial alignment of the articulating component may be changed to improve the ADR movement. The novel invention allows surgeons to change the axial alignment of an articulating component without changing the axial alignment of the ADR EP. This embodiment may also be used to customize an ADR to fit abnormal vertebrae.

FIG. 14A is an exploded coronal cross section of an alternative embodiment of an ADR, wherein the axial alignment of the convex component may be fixed as shown in FIG. 13A. The axial alignment of the modular concave component adjusts to fit the axial alignment of the convex component. FIG. 14B is an exploded coronal cross section of an alternative embodiment of the ADR drawn in FIG. 14A. Teeth from modular concave component 1402 cooperate with teeth in the lower ADR EP to prevent axial rotation between the components. This embodiment of the invention allows surgeons to adjust and fix the axial rotation of both articulating components relative to the ADR EPs. FIG. 14C is a view of the top of the convex component drawn in FIG. 13A, illustrating a hole 1406 that receives the screw and the teeth 1404 used to fix axial rotation between the component and the upper ADR EP. FIG. 14D is a view of the bottom of the concave articulating component drawn in FIG. 14B. The drawing illustrates the teeth 1402 that project from the sides of the component to fix the axial rotation between the articulating component and the lower ADR EP.

FIG. 15A is a coronal cross section of an alternative embodiment of the modular articulating components which have restricted axial rotation. The components may have articulating surfaces similar to the articulating surfaces drawn in FIG. 10A. FIG. 15B is an anterior view of the spine, the articulating components drawn in FIG. 15A, and the device drawn in FIG. 4H. In the preferred embodiments of the invention, the surgeon first measures the movements and forces required to produce the movements with the modular articulating components that allow unlimited axial rotation. The surgeon then measures the movements and forces required to produce the movements with the modular articulating components such as those drawn in FIG. 15A.

The axial rotation of the device with components with restrained axial rotation is adjusted until the movements and the forces to produce the movements are the same as those measured with the device with components that do not restrain axial rotation. The forces required to flex and extend the device are also minimized when the device is properly aligned. The vertebrae are marked to indicate the proper alignment of the final ADR (dotted areas of the drawing). This may then be used to align the ADR, such as that drawn in FIG. 10A. Surgeons may choose to use an alternative embodiment of the ADR, for example the ADR drawn in FIG. 12A, if they cannot align the device of FIG. 15B properly.

FIG. 16A is an anterior view of a trial ADR having modular articulating components similar to those drawn in FIG. 14A. The axial rotation between the modular articulating components is restricted. The modular articulating components enjoy unrestricted axial rotation relative to the upper and lower ADR EPs. The trial ADR is inserted after the vertebrae are machined to receive the keels of the ADR. The vertebrae may be machined to receive projections from the ADR EPs. The vertical lines mark the starting axial alignment of the four components. Compression and/or distraction instruments are used to move the implanted trial ADR through several cycles of flexion and extension. If the marks on the four components remain aligned after several cycles of flexion and extension, the surgeon has properly machined the vertebra to receive the ADR with the proper axial alignment.

FIG. 16B is an anterior view of the trial ADR drawn in FIG. 16A. The articulating components have rotated relative to the ADR EPs. For example, the articulating components may rotate after several cycles of flexion and extension, if the trial ADR was inserted with improper axial alignment. The figure indicates the articulating components move better if they are rotated a few degrees relative to the ADR EPs. Surgeons may choose to use the embodiment of the ADR EP drawn in FIG. 14B when the trial ADR indicates mal-alignment of the machined slots in the vertebrae. Alternatively, surgeons may chose to use an ADR without restriction, if the trial ADR indicates mal-alignment of the machined slots in the vertebrae.

FIG. 17A is an axial cross section through the upper vertebral endplate (VEP) of a vertebra. The image is similar to the axial images of CT scans and MRI scans. The dotted lines show one method of measuring the axial rotation of the disc. A line 1702 is drawn perpendicular to a line 1704 drawn across the posterior border of the vertebral body 1706. The angle formed (x) between the perpendicular line and a vertical line 1708 indicates the axial rotation of the disc space. The vertical line represents a line perpendicular to the floor. The CT scan is obtained with the patient lying flat on their back. Other methods could be used to measure the pre-operative axial rotation of the disc, for example lines could be drawn along the sides of the vertebra, the front of the vertebra, or between the pedicles. The pre-operative measurements, coupled with the device drawn in FIG. 9F help surgeons insert ADRs with the proper axial rotation.

FIG. 17B is an axial cross section of a vertebra. A guide pin 1720 has been placed into the anterior portion of the vertebra 1722. This embodiment of the invention incorporates the teachings in my co-pending U.S. Patent Application Ser. No. 60/519,405, incorporated herein by reference. For example, an intra-operative CT scan image may be used to determine the axial alignment of the disc the damaged disc. Intra-operative CT may also be used to align the guide pin inserted into the vertebra. Intra-operative CT or fluoroscopy may also be used to verify the anterior-to-posterior placement and the left to right placement of the ADR, ADR trial, or ADR cutting/machining guides relative to the VEPs. Intra-operative CT, MRI, or Fluoroscopy may also be used measure the size of the ADR, ADR trial, or ADR cutting/machining guides relative to the VEPs. ADRs that are placed to the left or right of midline, or that are placed to far anterior do not move as easily.

FIG. 17C is an anterior view of the spine with four alignment pins 1, 2, 3, 4 inserted into the vertebrae. The pins were inserted with the aid of intra-operative imaging as described in the text of FIG. 17B. The instruments use to insert the ADR trials, to insert the ADRs, and to machine the vertebrae may be aligned with the guide pins. FIG. 17D is an anterior view of one embodiment of an instrument that cooperates with the guide pins drawn in FIG. 17C. The guide is used to cut the slots into the vertebrae. The vertebrae are machined to receive ADR with keels. The cutting guide fits over the outer set of guide pins. The inner set of guide pins is removed from the vertebrae. The pins fit through the holes in the top and bottom of the guide. The elongated central opening in the guide is designed to guide a saw blade. The pins and the guide cooperate to assure the keel slots are cut into the vertebrae with proper axial alignment.

FIG. 18 is an anterior view of the spine and an alternative embodiment of the invention with modular articulating components that restrict axial rotation. Pressure transducers 1802, 1804 are placed on either side of the articulating components. The pressure transducers measure lateral bending of the device. The device should not bend laterally with flexion and extension, if the device is inserted with proper axial alignment. Alternatively, the lateral components could measure a change in distance rather than a change in pressure.

FIG. 19A is a superior view of an ADR EP wherein the keel 1902 of the ADR rotates about an axle 1904 in or near the center of the device. Pins or screws are placed into the holes on either side of the keel at ends of the keel. The pins or screws lock the keel in position. The angle of the keel relative to the ADR EP may be changed to adjust for mal-alignment of the slots cut into the vertebrae. FIG. 19B is a superior view of the ADR EP drawn in FIG. 19A. The keel is locked in a different position than the keel of the ADR EP drawn in FIG. 19A. FIG. 19C is a superior view of an ADR EP of an alternative embodiment of the invention wherein the keel swivels about an axle at one end of the keel. FIG. 19D is a superior view of the ADR EP drawn in FIG. 19C. The keel is locked in a different position than the keel of the ADR drawn in FIG. 19C.

We claim:

1. Apparatus to assess spinal movement, comprising:
a device adapted for physical connection to upper or lower vertebral bodies; and
one or more handles coupled to the device enabling a user to move one or both of the vertebral bodies to assess relative position, motion, or orientation.

2. The apparatus of claim 1, wherein the device is adapted for placement within an intradiscal space between the upper and lower vertebral bodies.

3. The apparatus of claim 1, wherein the device is externally expandable within an intradiscal space to reduce shear stress on the upper and lower vertebral bodies during insertion.

4. The apparatus of claim 3, wherein the device is externally expandable using one or more scissor jacks.

5. The apparatus of claim 1, wherein the device includes:
a pair of opposing endplates, each bearing against a respective one of the vertebral bodies; and
a set of modular articulating components situated between the opposing end plates facilitating a range of motion.

6. The apparatus of claim 5, further including a set of trial spacer components indexed to the modular articulating components to determine which set of the modular articulating components to utilize.

7. The apparatus of claim 5, wherein the modular articulating components include a feature to restrict motion in one or more dimensions.

8. The apparatus of claim 1, wherein the device is an artificial disc replacement (ADR).

9. The apparatus of claim 1, further including an instrument coupled to the device for measuring one or more aspects of the movement.

10. The apparatus of claim 9, wherein the instrument is coupled to the device between a pair of handles.

11. The apparatus of claim 9, wherein the instrument measures one or more distances associated with the movement.

12. The apparatus of claim 9, wherein the instrument measures the force required for a movement.

13. The apparatus of claim 9, wherein the instrument measures distances or forces associated with spinal flexion.

14. The apparatus of claim 9, wherein the instrument measures distances or forces associated with spinal extension.

15. The apparatus of claim 9, wherein the instrument measures distances or forces associated with lateral bending.

16. The apparatus of claim 9, wherein the instrument measures distances or forces associated with spinal rotation.

17. The apparatus of claim 9, wherein the instrument measures distances or forces associated with spinal retraction.

18. The apparatus of claim 17, wherein the instrument is a torque wrench or torque screwdriver.

19. The apparatus of claim 1, wherein the device includes a level to determine spinal alignment.

20. The apparatus of claim 19, wherein the device includes a handle with a level to determine spinal alignment.

21. The apparatus of claim 19, wherein the handle is oriented perpendicular to a patent in a prone position to determine rotational alignment.

22. The apparatus of claim 1, further including the use of pre-operative or intra-operative CT, MRI, or fluoroscopy to assist with the assessment of the relative motion or position.

23. The apparatus of claim 22, further including a guide for cutting slots into a vertebral body to receive a keel.

24. The apparatus of claim 22, further including an artificial disc replacement (ADR) with a rotatable keel to adjust for axial rotation.

25. An artificial disc replacement (ADR) with a rotatable keel to adjust for axial rotation.
